(19) United States
(12) Patent Application Publication
(10) Pub. No.: US 2024/0270504 A1
(43) Pub. Date: Aug. 15, 2024

(54) INSPECTION AND HANDLING SYTEMS AND METHODS

(71) Applicant: Cargill, Incorporated, Wayzata, MN (US)

(72) Inventors: Steve Butterworth, Hereford (GB); Matt Rayment, Coventry (GB)

(21) Appl. No.: 18/568,443

(22) PCT Filed: Jun. 9, 2022
(86) PCT No.: PCT/US2022/032851

§ 371 Date: Dec. 8, 2023

Related U.S. Application Data
(60) Provisional application No. 63/209,547, filed on Jun. 11, 2021.

(51) Int. Cl. B65G 43/08; B25J 11/00; B65G 47/00; G01N 33/12

(52) U.S. Cl. CPC B65G 43/08; B25J 11/0045; B65G 47/00; G01N 33/12; B65G 2201/0202; B65G 2203/041

(57) ABSTRACT

A method of inspecting and removing or repositioning products (14) on a moving conveyor (12) is described, including the step of moving an inspection and handling system (10) to a first location, wherein the inspection and handling system (10) includes a robot system (30) with an end effector (50), and wherein the inspection and handling system (10) is positionable relative to a camera system adjacent to the first location of the inspection and handling system (10). The method may further includes steps of conveying products (14) until they are adjacent to the camera and inspection and handling system (10), visually inspecting the products (14) and identifying a first unacceptable product (14), relaying a location of the first unacceptable product (14) to the robot system (30), moving the end effector (50) to remove the product (14) from the conveyor (12), and depositing the product (14) at a different location from the location from which it was removed.

### CROSS-REFERENCE TO RELATED APPLICATIONS

This application claims the benefit of U.S. Provisional Patent Application No. 63/209,547, filed 11 Jun. 2021, which is hereby incorporated by reference in its entirety.

### TECHNICAL FIELD

The present disclosure generally relates to a movable, multi-application automated system for visually inspecting and removing individual food items from a moving conveyor, and more particularly relates to inspecting food items (e.g., nuggets or other food items) that do not meet certain specifications and removing those nuggets from the moving conveyor for further processing and/or disposal.

### BACKGROUND

Individual food items are often partially processed for supplying to restaurants, such as fast food restaurants, where the final cooking of the food items is completed immediately prior to being served to a customer. For example, food items can be processed from various ingredients, cut or shaped into individual pieces, coated with one or more outer layers, partially pre-cooked, frozen, and/or otherwise processed and packaged so that restaurant employees only need to cook the items for a relatively brief period of time before they are ready to be served to the customer. This type of system helps to standardize the quantity and quality of cooked products that are provided to a consumer, along with improving food quality and reducing complaints.

Chicken nuggets are particularly popular food items that are provided in such a manner. In an exemplary process, chicken meat is processed and formed into individual shapes or nuggets, and then the nuggets are conveyed to another area for further processing, such as covering the nuggets with one or more coating layers, for example. The nuggets can then be conveyed to a pre-cooking station and/or conveyed to a packaging station, depending on the customer requirements. The nuggets can therefore be conveyed multiple times along different conveyors throughout the processing thereof.

When the nuggets are being conveyed, one or more inspectors are typically positioned adjacent to the conveyor to pick out and remove nuggets that do not meet all of the criteria necessary for further processing. For example, two or more nuggets 100 may be overlapping each other, such as is illustrated in FIG. 1a, a nugget 102 may have a non-meat substance or “contaminant” present on its outer surface, such as is illustrated in FIG. 1b, and/or a nugget 104 may be the incorrect shape or size, such as is illustrated in FIG. 1c. In any of these situations, the faulty nugget will be identified and manually removed by one of the inspectors for further processing and/or disposal. While such systems can be relatively effective, they require the complete attention of the inspector at all times, which can be somewhat tedious and repetitive and can therefore result in some faulty nuggets mistakenly being passed to the next step of the process. In addition, the conveyor speed when using this type of inspection is limited to the speed at which inspectors can view and accurately inspect the nuggets moving along the conveyor.

There is therefore a desire to provide a mobile automated system for inspecting food items on a moving conveyor that improves accuracy and minimizes human error. Such a system will desirably be capable of quickly removing food items that are identified as faulty from the conveyor and moving them to a predetermined location where they can be disposed of or reintroduced into the production cycle. It is further desirable that such a system is moveable and programmable for use at multiple locations within a production facility.

### SUMMARY

This disclosure generally relates to moveable systems that can interact with people and/or robots or other automation systems for increasing the automation of production sites, and particularly relates to the use of such systems in the food industry. The robot systems are designed to inspect and remove food items that are determined to be outside of predetermined specifications, such as products that are partly formed, incorrectly formed, broken, misshapen, overlapping, or otherwise determined to be unacceptable.

In one aspect of this invention, a method is provided for inspecting and removing or repositioning products on a moving conveyor with a mobile inspection and handling system. The method includes the steps of moving the inspection and handling system to a first location relative to the conveyor, wherein the inspection and handling system comprises a robot system extending from a frame, wherein the robot system comprises an end effector adjacent to a distal end of an arm system, and wherein the inspection and handling system is positionable relative to a camera system that is adjacent to the first location of the inspection and handling system. The method also includes conveying multiple products on the conveyor until the products are adjacent to the camera system and inspection and handling system, visually inspecting each of the multiple products with the camera system and identifying a location of a first unacceptable product, relaying the location of the first unacceptable product to the robot system, moving the end effector to remove the first unacceptable product from the conveyor, depositing the first unacceptable product at a different location from the location from which it was removed, and repeating the visual inspection step, relaying location information step, moving the end effector step, and depositing the product step for each subsequent unacceptable product.

The method may further include a step of moving the inspection and handling system to a second location after a last unacceptable product is removed from the conveyor. The step of depositing the first unacceptable product at a different location from where it was removed may include depositing the first unacceptable product at a different location on the conveyor or depositing the first unacceptable product at a location that is not on the conveyor.

The inspection and handling system may include at least one of multiple casters and multiple wheels extending from a lower member of the frame. The frame may also include leveling members, such that the method also includes leveling the inspection and handling system after the step of moving the inspection and handling system to the first location. The inspection and handling system may be moveable with at least one of a fork lift mechanism, a pallet truck mechanism, and manual manipulation.

The end effector may include at least one gripper member, which may be removable and replaceable. The conveyed products can include food products, and more specifically can include formed food products, and even more specifically can include chicken nuggets.

Prior to the step of moving the inspection and handling system to the first location, the method may further include a step of programming at least one of the robot system and the camera system to detect unacceptable products.

The method may further include a step of positioning the camera system relative to the conveyor either before or after the step of moving the inspection and handling system to the first location relative to the conveyor. The method may also include a step of repeatedly moving the inspection and handling system to multiple locations relative to the conveyor. The method may also include a step of securing the inspection and handling system relative to the conveyor after it has been moved to a first location and subsequent locations.

In another aspect of the invention, a mobile inspection and handling system is provided for inspecting and removing or repositioning products on a conveyor which includes a robot system extending from a frame, wherein the robot system comprises an end effector adjacent to a distal end of an arm system, wherein the robot system is programmable for use at multiple locations relative to a conveyor. The frame may further include at least one of a wheel or caster extending from a lower frame member, and may further include a camera system for visually inspecting multiple products and identifying a location of each unacceptable product. The mobile inspection and handling system may further include a location system for positioning the inspection and handling system in a specific location.

### BRIEF DESCRIPTION OF THE DRAWINGS

The present disclosure will be further explained with reference to the appended Figures, wherein like structure is referred to by like numerals throughout the several views, and wherein;

FIGS. 1a-1c are top views of three different configurations of chicken nuggets that would be outside of specification for further processing:

FIG. 2 is a side view of an exemplary embodiment of an inspection and handling system;

FIG. 3 is a top view of the inspection and handling system of FIG. 2;

FIG. 4 is a perspective view of an exemplary embodiment of an inspection and handling system;

FIG. 5 is top view of the inspection and handling system of FIG. 4;

FIG. 6 is a front view of the inspection and handling system of FIG. 4;

FIG. 7 is a side view of the inspection and handling system of FIG. 4;

FIG. 8 is a perspective view of an exemplary embodiment of an inspection and handling system, without a safety shield;

FIG. 9 is a perspective view of the inspection and handling system of FIG. 8, including a safety shield;

FIG. 10 is a top view of the inspection and handling system of FIG. 9;

FIG. 11 is a front view of the inspection and handling system of FIG. 9;

FIG. 12 is a side view of the inspection and handling system of FIG. 9, with the safety shield removed so that a robot system is visible;

FIG. 13 is a perspective view of an exemplary embodiment of an inspection and handling system that includes two robot systems;

FIG. 14 is a top view of the inspection and handling system of FIG. 13;

FIG. 15 is a front view of the inspection and handling system of FIG. 13;

FIG. 16 is a front perspective view of a base unit for an inspection and handling system of the type illustrated in FIG. 13;

FIG. 17 is back perspective view of the base unit illustrated in FIG. 16;

FIG. 18 is an exemplary embodiment of a foot member that can be used with inspection and handling systems disclosed herein;

FIG. 19 is an enlarged view of a door cover with hinges of the base unit illustrated in FIG. 16;

FIG. 20 is a perspective view of an exemplary embodiment of a frame for use with a camera for an inspection and handling system;

FIG. 21 is a perspective view of an exemplary embodiment of a robot system for use with an end effector for an inspection and handling system;

FIG. 22 is a side view of an exemplary embodiment of a robot arm and end effector for an inspection and handling system;

FIG. 23 is a side view of an exemplary embodiment of a robot arm and end effector for an inspection and handling system;

FIG. 24 is a perspective view of an exemplary embodiment of an end effector:

FIG. 25 is a side view of the end effector illustrated in FIG. 24, with one visible gripper member;

FIG. 26 is a front view of the end effector illustrated in FIG. 24, with two visible gripper members:

FIG. 27 is a perspective view of an exemplary embodiment of an end effector;

FIG. 28 is an exploded view of the embodiment of an end effector illustrated in FIG. 27:

FIG. 29 is a perspective view of an exemplary embodiment of an end effector that includes a cover member that is transparent:

FIG. 30 is a bottom view of the end effector of FIG. 29; and

FIG. 31 is a side view of the end effector of FIG. 29.

### DETAILED DESCRIPTION

The present disclosure is directed to systems used for visually inspecting items being moved from one location to another via a conveyor belt or similar system, detecting items that do not meet certain specifications, and then removing those faulty items. One exemplary use of such systems is in the food processing industry, such as for use in the processing of nuggets (e.g., chicken nuggets). However, it is understood that the systems described herein can also be used for a variety of other food and non-food items.

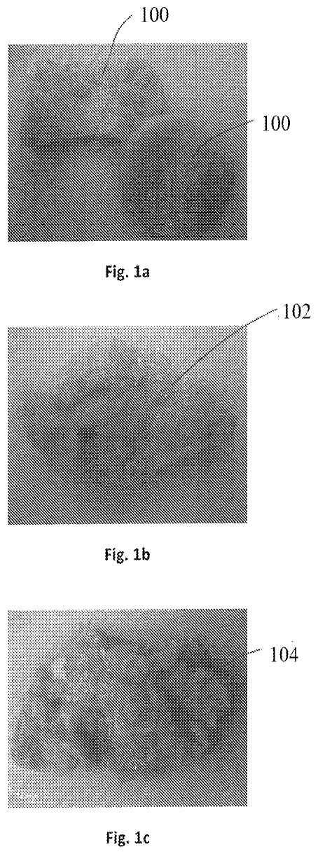

With regard to exemplary embodiments discussed herein, reference is again made to FIGS. 1a-1c, which illustrate three typical examples of situations where nuggets on a conveyor would be considered faulty and need to be removed from further processing. For example, FIG. 1a illustrates two nuggets 100 overlapping each other, FIG. 1b illustrates a nugget 102 having a non-meat substance or “contaminant” visible on its outer surface, and FIG. 1c illustrates a misshapen nugget 104. In any of these or other predetermined situations, the faulty nuggets will be identified and removed by the systems described herein for further processing and/or disposal. It is preferred that such a system allows for both quicker and more accurate inspection and removal of faulty products than the present manual inspection and removal processes.

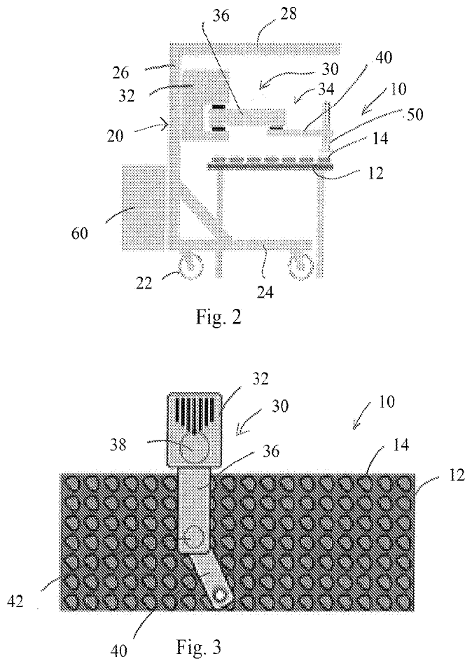

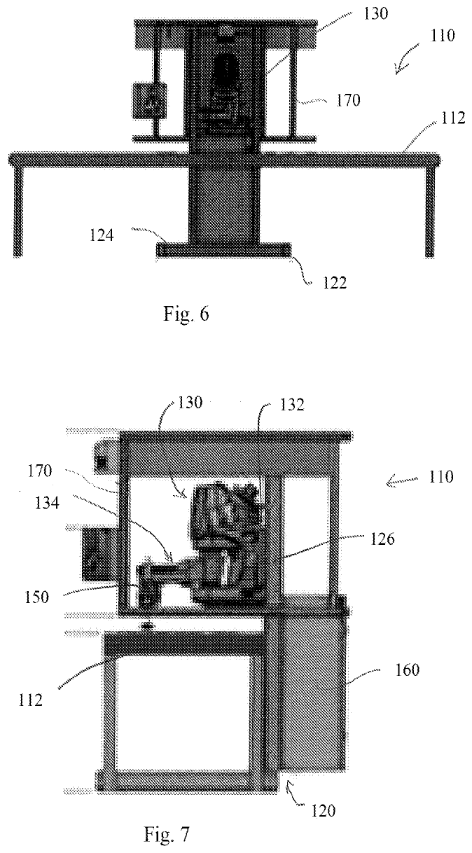

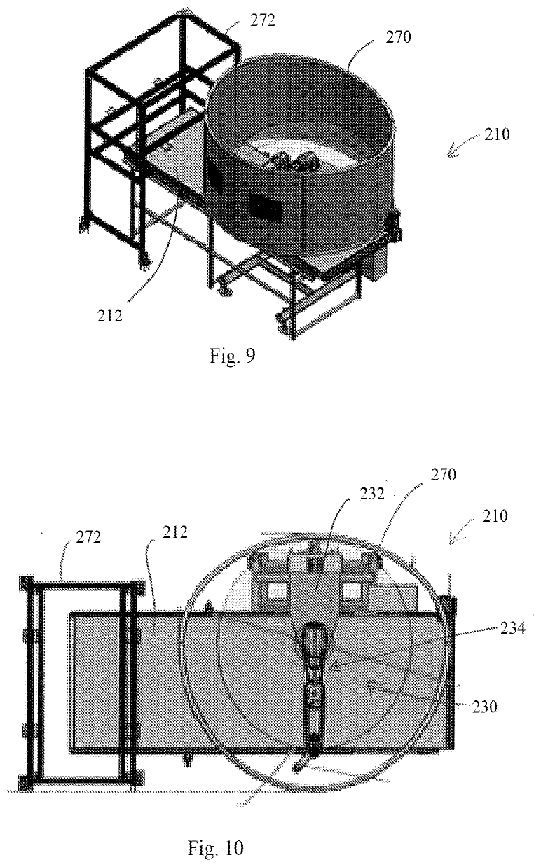

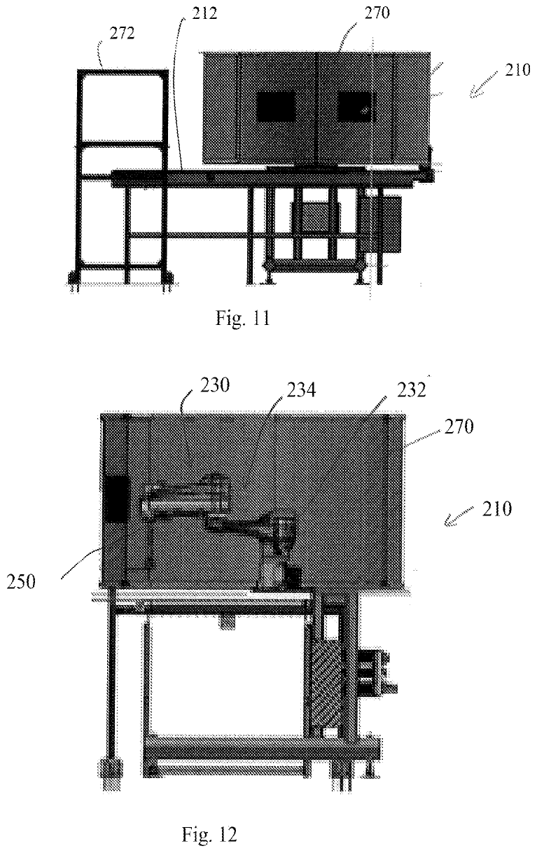

Referring now to FIGS. 2-15, wherein the components are labeled with like numerals throughout the several Figures, and initially to FIGS. 2 and 3, an exemplary configuration of an inspection and handling system 10 is illustrated relative to a conveyor 12 with multiple food items or nuggets 14 on its top surface. System 10 generally includes a frame 20 with wheels or casters 22 extending from a lower frame member 24. A generally vertical frame member 26 extends upwardly from lower frame member 24, and an upper frame member 28 extends outwardly from the vertical frame member 26 in generally the same direction in which the lower frame member 24 extends. The upper and lower frame members 24, 28 are generally configured to aid in the stability of the system 10 by preventing it from tipping during use or when being moved. As shown, the upper frame member 28 extends over the top of the conveyor 12 while the lower frame member 24 extends below at least a portion of the conveyor 12.

The system 10 further includes a robot system 30 that is attached to the frame 20 and is used for inspection of the conveyed items. Robot system 30 includes a base member 32 attached to the vertical frame member 26. An arm system 34 extends from the base member 32. Arm system 34 includes a first arm member 36 that is moveable about a first pivot area 38, and a second arm member 40 extending from the first arm member 36 and moveable about a second pivot area 42. The arm system 34 may include more or less than two arm members, depending on the range of motion, complexity, and speed desired for the inspection process.

The robot system 30 further includes an end effector 50 positioned generally at the distal end of the second arm member 40 that is opposite the second pivot area 42. The end effector 50 may have a wide variety of configurations, which will be discussed in further detail below. With regard to this system 10, however, it is understood that the end effector 50 will be used to remove selected nuggets from the conveyor in response to instructions provided a computer that is controlling the robot system 30.

The system 10 further includes a base unit 60 that is shown in this embodiment as extending from the opposite side of the vertical frame member 24 from the base member 32 of the robot system 30. Base unit 60 is positioned adjacent to the lower frame member 24 and can include a box or enclosure in which the electronics (i.e., computer) that control the robot system 30 are located. The base unit 60 can include one or more panels that pivot or move relative to each other to allow access to its inner area for programming or other adjustments to the computer. The base unit 60 can also include control features, such as knobs or dials, which are accessible by a user without having to access its inner area. The base unit 60 may also include display screens or other mechanisms by which operators can view information regarding operation of the robot system 30.

The system 10 further includes an imaging or camera system (not shown in FIGS. 2 and 3) that visually inspects the nuggets as they are being conveyed toward the arm system 34. The camera system may optionally include one or more lights directed toward the conveyor so that the flaw detection process can be conducted more effectively. The camera system will be programmable for detecting one or more different type of flaws or issues that are considered unacceptable. This information is conveyed to the robot system 30 that will then instruct the end effector 50 to move toward the flawed nugget for its removal from the conveyor. The end effector 50 will then be moved to a position where it will be able to remove additional nuggets from the conveyor when needed.

The wheels or casters 22 allow the system 10 to be moved to different locations, as desired, to perform the same or different procedures at different locations along the production line. In general, the system 10 is designed so that the arm system 34 will reach over selected conveyor locations on a production line, wherein the system 10 can be moved by wheeling it to the desired location(s). In the case of a nugget production system, the system 10 can be moved to locations such as the following: immediately after the nuggets are formed and being moved to the next processing stage: after the process of pre-dusting or powdering the surface of the nuggets: after the nuggets have been fried in a fryer; and/or after the nuggets have been frozen and are being conveyed to the packaging equipment. The robot system 30 will be programmable and have pre-written code that allows the camera to detect flaws that are specific to each of the processes, depending on where the system 10 is located in the overall production process.

The movable base unit 60 can utilize a male and female location system, for example, that is able to accurately position the system 10 relative to a conveyor, processing equipment, a specific floor location, or other equipment. The combination of and interaction between such systems can enable the automation equipment to be quickly and repeatedly located at precise, predetermined locations relative to the production line conveyor and/or other equipment. The location system can provide power, communications, compressed air, water, and the like to the robotic and camera systems. This system may also enable the robotic and camera system to connect to the conveyor to receive data such as speed, emergency stop signals, and the like. The location system can include digital identifying systems and processes for digitally confirming the relative position of the equipment.

The location system may also be used to physically connect or clamp the robot and vision systems securely in place relative to each other. The location system may be used to provide a predetermined separation between a robot system and a vision system. Communication between the location system and the production equipment (e.g., the conveyor) can act as an extension of the robotic and camera system to increase its safety and stability during operation.

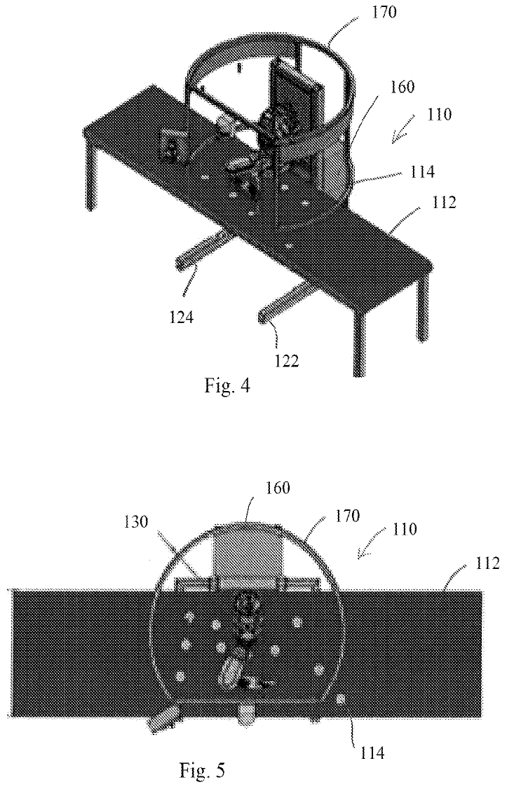

Referring now to FIGS. 4-7, an exemplary configuration of an inspection and handling system 110 is illustrated relative to a conveyor 112 with multiple food items or nuggets 114 on its top surface. System 110 generally includes a frame 120 with wheels or casters 122 extending from a lower frame member 124. As with other systems described herein, wheels or casters below the bottom of the frame 120 allow the system 110 to be moved to different locations, as desired, to perform the same, similar, or different procedures at different locations along the production line. A generally vertical frame member 126 extends upwardly from lower frame member 124. As shown, the lower frame member 124 extends below at least a portion of the conveyor 112. In particular, the conveyor 112 includes a relatively open space beneath it into which the lower frame member 124 can be positioned.

The system 110 further includes a robot system 130 that is attached to the frame 120 and is used for inspection of the conveyed items. As shown best in FIG. 7, robot system 130 includes a base member 132 adjacent to the vertical frame member 126. An arm system 134 extends from the base member 132. Arm system 134 includes one or more articulated arm sections, as desired to quickly reach each nugget that is to be removed from the conveyor 112. The robot system 130 further includes an end effector 150 positioned at the end of one of the arm sections. The end effector 150 may have a wide variety of configurations, which will be discussed in further detail below. With regard to this system 110, however, it is understood that the end effector 150 will be used to remove selected nuggets from the conveyor in response to instructions provided by a computer that is controlling the robot system 130.

System 110 further includes a safety shield 170 that extends around at least part of the area where the arm system 134 is able to move relative to the conveyor 112. The shield 170 can optionally include open areas or windows to allow a user to view the conveying process without physically interfering with the inspection and removal of nuggets.

The system 110 further includes a base unit 160 that is shown in this embodiment as extending from the opposite side of the vertical frame member 124 from the base member 132 of the robot system 130. The base unit 160 can also have cable or wire entry and exit locations to allow for the connection of a camera system and/or other electronic components used for tracking of the conveyor. Base unit 160 can include a box or enclosure in which the electronics (i.e., computer) that control the robot system 130 are located. The base unit 160 can also include control features, such as knobs or dials, which are accessible by a user without having to access its inner area. The base unit 160 may also include display screens or other mechanisms by which operators can view information regarding operation of the robot system 130. The system 110 further includes an imaging or camera system (not shown) that visually inspects the nuggets as they are being conveyed toward the arm system 134. Information obtained by the imaging or camera system will be processed to instruct the robot systems regarding the particular nuggets that are to be removed from the conveyor.

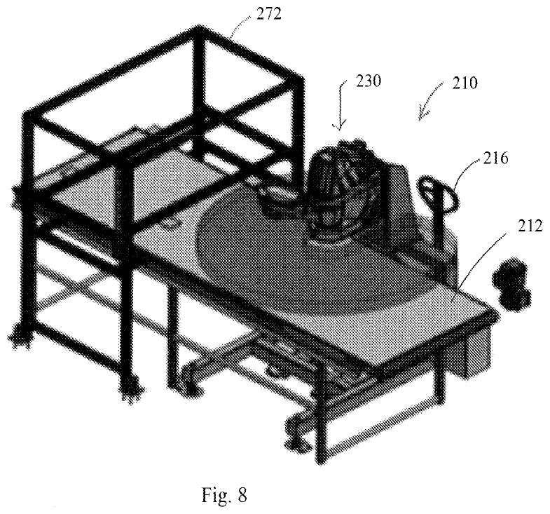

Referring now to FIGS. 8-12, an exemplary configuration of an inspection and handling system 210 is illustrated relative to a conveyor 212 used for moving multiple food items or nuggets on its top surface. System 210 is shown as being moveable with a manual or powered forklift or pallet truck system 216, although the system 210 can include a frame with wheels or casters, or can have a different support configuration. The forklift or pallet truck system 216 may be used to move the system 210 to different locations, as desired, to perform the same, similar, or different procedures at multiple locations along the production line. The forklift or pallet truck system 216 can remain engaged with the system 210 while it is being used for inspection and removal of products, or the forklift 216 or pallet truck system can instead deposit the system 210 in a desired location and then be moved away from the system 210 until it needs to be moved again.

The system 210 includes a robot system 230 that is attached to a frame 220 and is used for inspection of the conveyed items. Robot system 230 includes a base member 232 attached to a frame or other structure, and an arm system 234 extending from the base member 232. Arm system 234 includes one or more articulated arm sections, as desired, to quickly reach each nugget that is to be removed from the conveyor 212. The robot system 230 further includes an end effector 250 positioned at the end of the distalmost arm section. The end effector 250 may have a wide variety of configurations, which will be discussed in further detail below. With regard to this system 210, however, it is understood that the end effector 250 will be used to remove selected nuggets from the conveyor in response to instructions provided a computer that is controlling the robot system 230.

System 210 further includes a safety shield 270 (not shown in FIG. 8) that extends around at least part of the area where the arm system 234 is able to move relative to the conveyor 212. The shield 270 can include open areas or windows to allow a user to view the conveying process without physically interfering with the inspection and removal of nuggets. The system 210 may further include an imaging or camera system (not shown) that is attachable to a camera frame 272 or elsewhere relative to one of the processes to visually inspect the nuggets as they are being conveyed relative to the arm system 234.

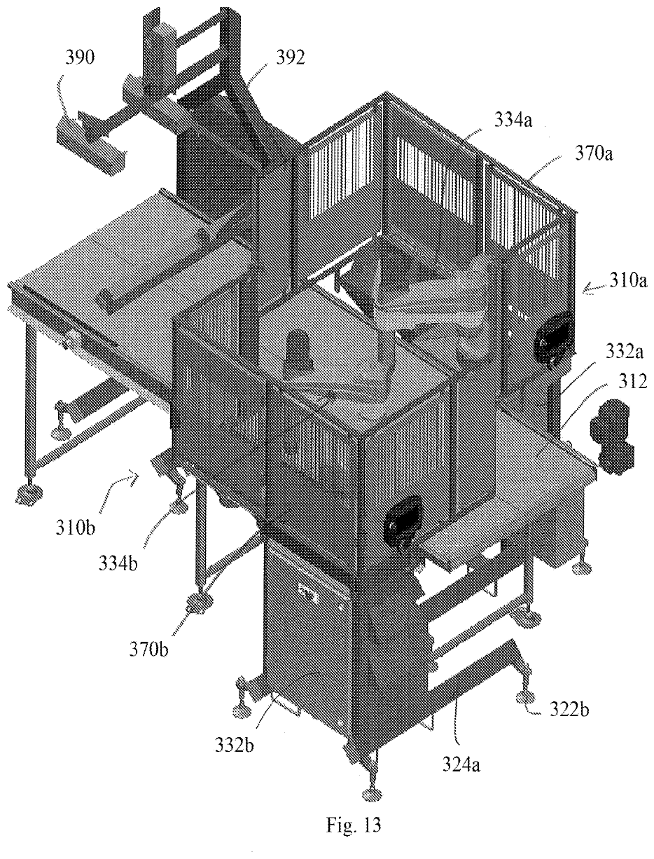

Referring now to FIGS. 13-15, exemplary configurations of a first inspection and handling system 310a and a second inspection and handling system 310b are positioned along opposite edges of a conveyor 312 used for moving multiple food items or nuggets on its top surface. System 310a generally includes a first base member 332a with feet (not visible) extending from its bottom members, and system 310b generally includes a first base member 332b with feet 322b extending from its bottom members 324b. The feet can optionally include adjustability features to allow for leveling of the systems, as desired. Alternatively or additionally, the systems 310a, 310b can include wheels or casters extending from bottom members and contacting the floor. As with other systems described herein, wheels or casters can allow the systems 310a, 310b to be moved to different locations, as desired, to perform the same or different procedures at different locations along the production line. When wheels or casters are not provided, the systems 310a, 310b can be moved using a fork lift or other mechanism, for example.

Although the systems 310a, 310b are shown to be identical or similar to each other in these figures, it is understood that two or more systems can be used that are configured differently from each other. In cases where multiple systems are used relative to the same conveyor belt, the systems can be configured to detect the same or different features of the items moving along the conveyor.

Systems 310a, 310b each include a robot system 330a, 330b, respectively, attached to respective base members 332a, 332b so that they can be used for inspection of conveyed items. Robot systems 330a, 330b each include a respective arm system 334a, 334b extending from base members 332a, 332b. Arm systems 334a, 334b each include one or more articulated arm sections, as desired, to quickly reach each nugget that is to be removed from the conveyor 312. The robot systems 330a, 330b each further include an end effector 350a, 350b, respectively, positioned at the end of one of the arm sections. The end effectors 350a, 350b may have a wide variety of configurations. With regard to systems 310a, 310b, however, it is understood that the end effectors 350a, 350b will be used to remove selected nuggets from the conveyor in response to instructions provided a computer that is controlling the robot systems 330a, 330b.

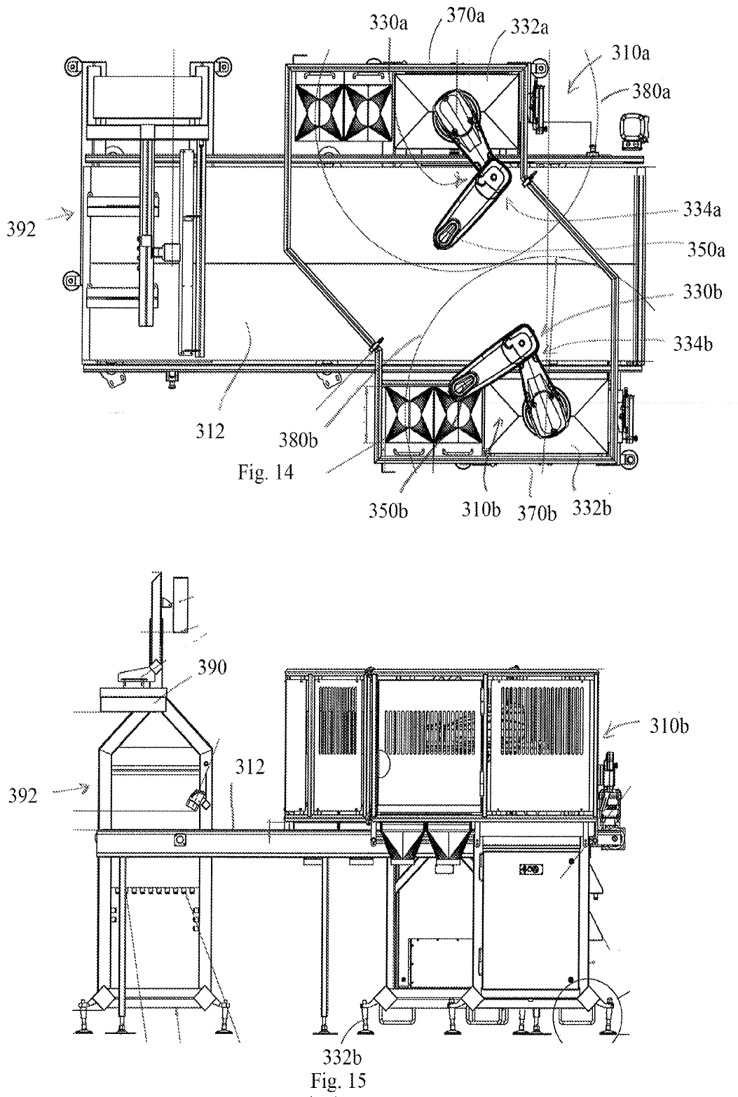

When two systems are used, such as systems 310a, 310b of FIGS. 13-15, the systems can be designed such that their ranges of motion of their arm systems 334a, 334b do not intersect with each other, as is illustrated by adjacent circles 380a, 380b of FIG. 14. Additionally or alternatively, the systems 310a, 310b are programmed so that the arm systems 334a, 334b will not contact each other while operating.

Systems 310a, 310b each include a safety shield 370a. 370b that extends around at least part of the area where the arm systems 334a, 334b are able to move relative to the conveyor 312. The shields 370a, 370b can include open areas or windows to allow a user to view the conveying process without physically interfering with the inspection and removal of food items. The systems 310a, 310b may further include an imaging or camera system 390 that is attachable to a camera frame 292 or elsewhere relative to one of the processes to visually inspect the nuggets as they are being conveyed along the conveyor 312.

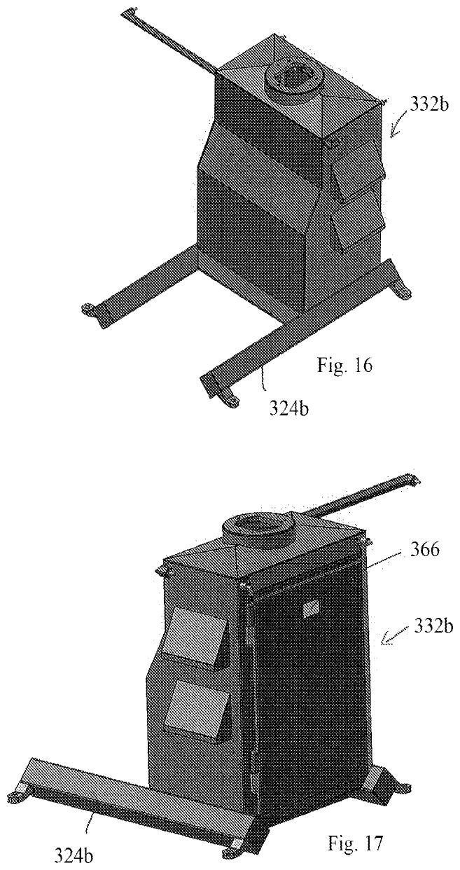

FIG. 16 is a front perspective view of base unit 332b for an inspection and handling system such as system 310b illustrated in FIG. 13, and FIG. 17 is a back perspective view of the base unit 332b. As shown, base unit 332b can include a box or enclosure in which the electronics (i.e., computer) that control a robot system are located, and lower frame members 324a extending from its bottom side. The base unit 332b can also include control features, such as knobs or dials 366, an exemplary configuration of which is illustrated in the enlarged view of FIG. 19. These control features are accessible by a user without having to access the inner area of the base unit 360. FIG. 19 also illustrates an optional hinged door for accessing the interior portion of the base unit, such as to adjust or otherwise modify that electronics that control the robot. The base unit 332b may also include display screens or other mechanisms by which operators can view information regarding operation of a corresponding robot system.

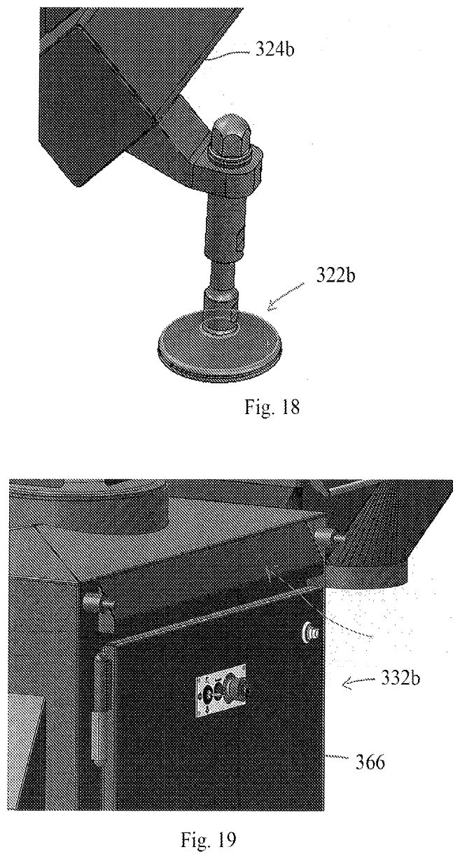

FIG. 18 is an enlarged view of an exemplary embodiment of a foot member 332b extending from a lower frame member 324a. Such a foot member 332b can swivel and/or have features to allow it to be adjusted vertically to level the base unit from which it extends, as desired.

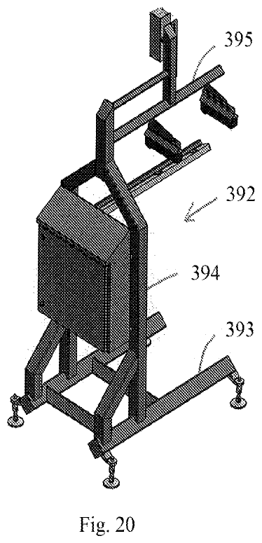

FIG. 20 is a perspective view of an exemplary embodiment of camera frame 392 for use with one or more cameras for an inspection and handling system. The frame 392 includes a generally vertical frame structure 394 extending upwardly from lower frame member 393, and an upper frame member 395 extending outwardly from the vertical frame structure 394 in generally the same direction in which the lower frame member 393 extends. The upper and lower frame members 393, 395 are generally configured to aid in the stability of the system camera frame 392 by preventing it from tipping during use or when being moved. As shown, the upper frame member 395 can extend over the top of a conveyor while the lower frame member 393 can extend below at least a portion of that conveyor.

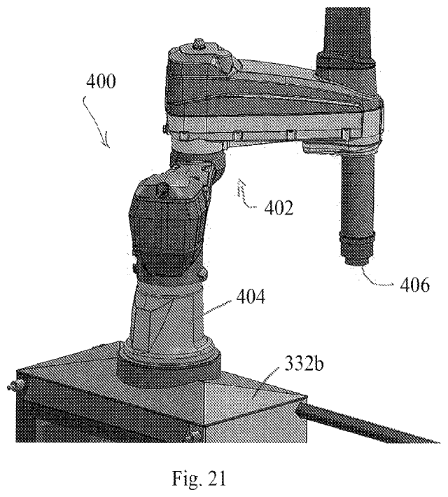

FIG. 21 is a perspective view of an exemplary embodiment of a robot system 400 for use with an end effector for an inspection and handling system, such as embodiments of such systems described herein. The robot system 400 can be mounted on a base unit similar to the base unit 332b illustrated in FIGS. 16 and 17, for example, so that it can be moved to one or more locations relative to a conveyor. The robot system 400 generally includes a moveable arm system 402 extending from a robot base member 404. The arm system 402 includes an end effector 406 at its distal end, which is configured to pick up items from a moving conveyor.

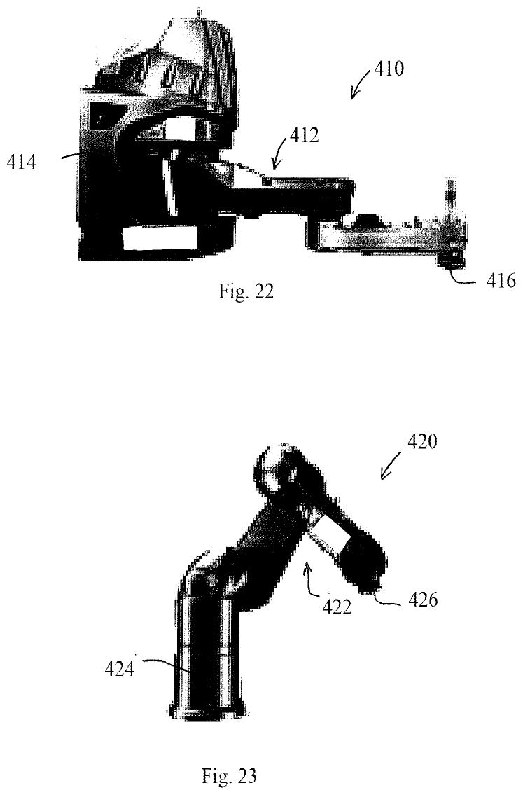

FIGS. 22 and 23 illustrate two additional exemplary embodiments of robot systems 410, 420. Each of these systems can be mounted to a base unit or other structure so that they are positionable relative to one or more locations along a conveyor. The robot systems 410, 420 include moveable arm systems 412, 422, respectively, that extend from respective robot base members 414, 424. The arm systems 412, 422 include end effectors 416, 426, respectively, at their distal ends, which are configured to pick up items from a moving conveyor. As shown, arm system 412 of FIG. 22 includes arm sections that are pivotable relative to each other along vertical axes, but generally move in a single horizontal plane when in use. Arm system 422 includes arm sections that can be considered to “articulate” or move in multiple planes relative to each other when in use.

The use of the term “end effector” herein refers generally to the portion of a robot system at its distal end that comes into contact with the items on a conveyor (e.g., food nuggets) that are to be picked up for removal from the production line. The end effectors discussed herein can have a wide variety of configurations, but generally can include a gripper end that is capable of picking up single items with visible contaminants, items that are partly formed or incorrectly formed or broken, items that are misshapen, overlapping items, or adjacent items that are too close and/or contacting each other. The end effectors can remove such items from the production line for further processing or disposal, and/or can reposition items on the conveyor, such as in the cases where the items are formed correctly but are overlapping or too close to each other.

The gripper end of the end effectors described herein are generally designed to be able to remove items from a conveyor without unintentionally dropping the items onto the conveyor during transportation. The gripper end can also be designed such that it can be used in many different stages of the production of the nuggets. For example, the gripper end can be capable of picking up items after their formation, after they have been coated, after the items have been fried, and/or after the items have been frozen. The gripper end can be designed to pick up and release product on items on command through the operation of switching the gripper end on and off, respectively.

The gripper end should be capable of functioning without operator intervention for extended periods of time, and therefore can include mechanisms to keep its components from clogging. That is, the gripper end is configured to be capable of operating without causing food safety hazards, product quality issues, or interruption to the production line (e.g., contamination added to the conveyor, unscheduled downtime, and the like).

When the gripper end requires cleaning, one or more components of the end effector can include quick-change connections to allow for easy removal and replacement/reconnection. In order to minimize contamination, however, the gripper end is generally designed to limit the ingress of microorganisms, and can also be designed with minimal external crevices and sharp corners that are difficult to clean. In certain embodiments, the end effector and other components can be made from stainless steel and plastics.

Certain portions of the end effector can also be made to be disposable to eliminate the downtime that may be required to clean certain components. In certain cases, it may be beneficial for the entire end effector to be removable and replaceable, either for maintenance and cleaning purposes or in cases where it is desired to use different end effectors for different locations or processes of a particular production line. In other cases, components such as the gripper ends can be removable and replaceable.

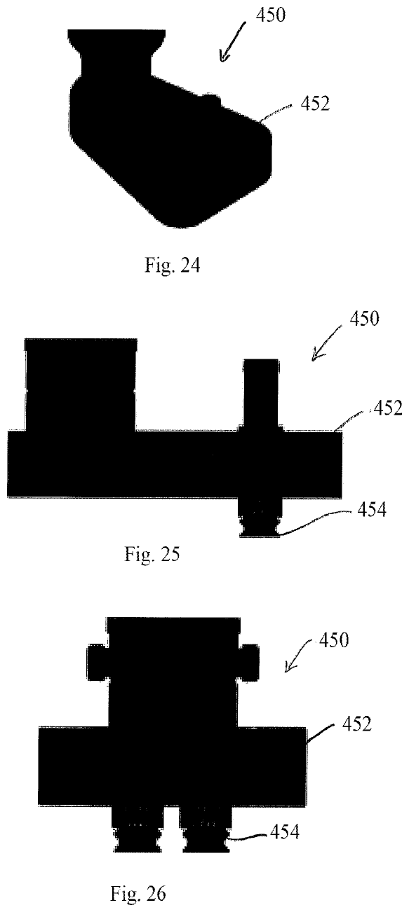

FIGS. 24-26 provide a perspective view, a side view, and a front view, respectively, of an exemplary embodiment of an end effector 450. End effector 450 includes a plate 452 with a lower surface from which gripper members 454 extend. Although the illustrated end effector 450 includes two of such gripper members 454, it is also possible for the end effectors used with the systems provided herein to include more or less than two gripper members. The gripper members 454 are considered to be “flow grippers” that generally operate via a vacuum that turns on to pull items toward its surface and then turns off to release or drop the items once the robot system has moved the items to their desired location.

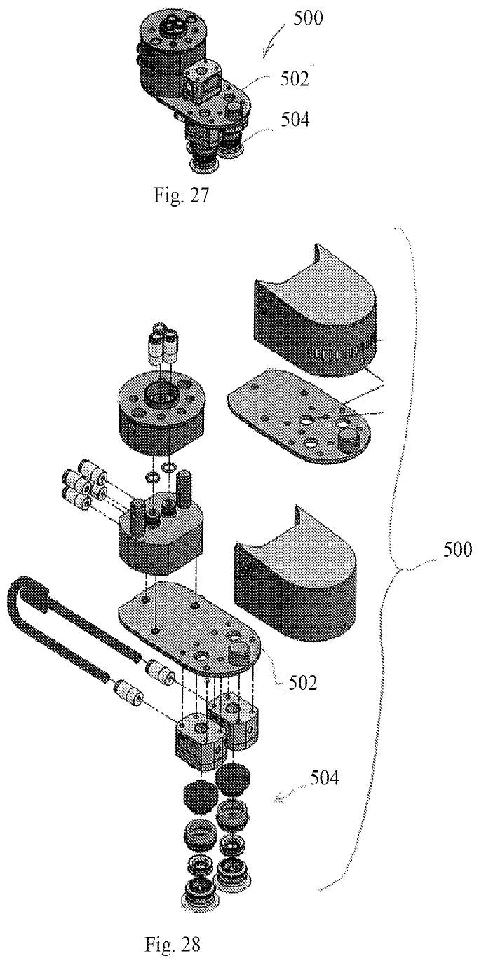

FIGS. 27 and 28 provide a perspective view and an exploded view, respectively, of an exemplary embodiment of an end effector 500. End effector 500 includes a plate 502 with a lower surface from which gripper members 504 extend. Although the illustrated end effector 500 includes two of such gripper members 504, it is also possible for the end effectors used with the systems provided herein to include more or less than two gripper members 504.

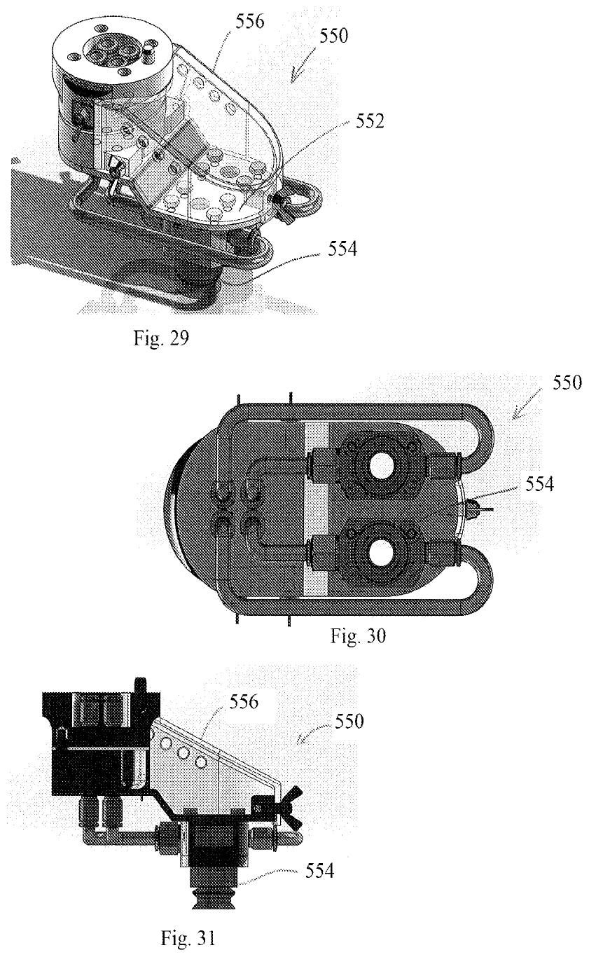

FIGS. 29-31 provide a perspective view, a bottom view, and a side view, respectively, of an exemplary embodiment of an end effector 550. End effector 550 includes a plate 552 with a lower surface from which gripper members 554 extend. Although the illustrated end effector 550 includes two of such gripper members 554, it is also possible for the end effector 550 to include more or less than two gripper members 554. End effector 550 further includes a cover 556 that can be transparent, translucent, or opaque, although a transparent cover 556 provides the ability to view the area above the gripper members 554 during operation of the end effector 550.

As described herein, the inspection and handling systems are used to interact with people and/or to interact with other robots. More broadly, the robot systems can be used to interact with other automation in the inspection and removal of defective or improperly placed items from a conveyor. In more particularity, the systems are configured for use in food production automation applications.

Embodiments of the inspection and handling systems described herein are configured to be mobile or portable, either via wheels or casters on the frame of the system so that an operator can manually position it in a desired location relative to a conveyor, or via a pallet truck or fork lift truck that engages the inspection and handling system to move it between locations. In some cases, it may be desirable to provide foot members extending from the bottom of a frame in addition to or instead of wheels or casters, wherein such foot members will contact the floor and support the inspection and handling system. An example of such a foot member is described above relative to FIG. 18, which illustrates the foot member 332b extending from a lower frame member 324a. Such a foot member can swivel and/or have features to allow it to be adjusted vertically to level the base unit from which it extends, as desired. The system can also be provided with leveling features that automatically adjust the foot members or other leveling structures to level the base of the system that holds the robot. Thus, it is contemplated that indicators can be incorporated into a portion of the system that provides feedback to the leveling system to adjust the leveling when needed.

It is further contemplated that the inspection and handling systems described herein can be moved to desired locations via an autonomous intelligent vehicle. Such a vehicle can be self-guiding and self-navigating such that it is capable of moving around the production facility to desired locations without manual intervention.

As is discussed above, embodiments of the inspection and handling systems described herein also include an imaging or camera system for visually inspecting items as they are being moved along a conveyor toward robot arms that can remove and/or relocate defective items. The camera system may optionally include one or more lights and will generally be programmable for detecting one or more different type of flaws or issues with the items. Because the arm system of the robot system will be moving to reach conveyed items with its end effector, it can generally be advantageous for the camera system to be mounted on its own frame that is separate from the robot system to provide independent stability to both the camera system and the robot system. However, it is possible for the camera system to be mounted on the same frame as the robot system. In either case, the camera system will be positioned prior to the robot system relative to the direction the items are being conveyed so that the information regarding defective products can be sent to the robot system. The end effector will then be moved to those item(s) for removal thereof. It is contemplated that one or more cameras may be mounted relatively close to the conveyor to inspect the height of the items and/or overlap that may be difficult to detect with the overhead cameras.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A method of inspecting and removing or repositioning products on a moving conveyor with a mobile inspection and handling system, the method comprising the steps of:
moving the inspection and handling system to a first location relative to the conveyor, wherein the inspection and handling system comprises a robot system extending from a frame, wherein the robot system comprises an end effector adjacent to a distal end of an arm system, and wherein the inspection and handling system is positionable relative to a camera system that is adjacent to the first location of the inspection and handling system;
conveying multiple products on the conveyor until the products are adjacent to the camera system and inspection and handling system;
visually inspecting each of the multiple products with the camera system and identifying a location of a first unacceptable product;
relaying the location of the first unacceptable product to the robot system;
moving the end effector to remove the first unacceptable product from the conveyor;
depositing the first unacceptable product at a different location from the location from which it was removed; and
repeating the visual inspection step, relaying location information step, moving the end effector step, and depositing the product step for each subsequent unacceptable product.

2. The method of claim 1, further comprising a step of moving the inspection and handling system to a second location after a last unacceptable product is removed from the conveyor.

3. The method of either of claim 1, wherein the step of depositing the first unacceptable product at a different location from where it was removed comprises depositing the first unacceptable product at a different location on the conveyor.

4. The method of claim 1, wherein the step of depositing the first acceptable product at a different location from where it was removed comprises depositing the first unacceptable product at a location that is not on the conveyor.

5. The method of claim 1, wherein the inspection and handling system comprises at least one of multiple casters and multiple wheels extending from a lower member of the frame.

6. The method of claim 1, wherein the frame includes leveling members, and wherein the method further comprises a step of leveling the inspection and handling system after the step of moving the inspection and handling system to the first location.

7. The method of claim 1, wherein the end effector comprises at least one gripper member.

8. The method of claim 7, wherein the at least one gripper member is removable and replaceable.

9. (canceled)

10. The method of claim 1, wherein the conveyed products comprise formed food products.

11. The method of claim 1, wherein the formed food conveyed products comprise chicken nuggets.

12. The method of claim 1, wherein prior to the step of moving the inspection and handling system to the first location, the method further comprises a step of programming at least one of the robot system and the camera system to detect unacceptable products.

13. The method of claim 1, wherein the inspection and handling system is moveable with at least one of a fork lift mechanism, a pallet truck mechanism, and manual manipulation.

14. The method of claim 1, further comprising a step of positioning the camera system relative to the conveyor either before or after the step of moving the inspection and handling system to the first location relative to the conveyor.

15. The method of claim 1, further comprising a step of repeatedly moving the inspection and handling system to multiple locations relative to the conveyor.

16. The method of claim 1, further comprising a step of securing the inspection and handling system relative to the conveyor after it has been moved to a first location and subsequent locations.

17. A mobile inspection and handling system for inspecting and removing or repositioning products on a conveyor, the system comprising:
a robot system extending from a frame, wherein the robot system comprises an end effector adjacent to a distal end of an arm system,
wherein the robot system is programmable for use at multiple locations relative to a conveyor.

18. The mobile inspection and handling system of claim 17, wherein the frame comprises at least one of a wheel or caster extending from a lower frame member.

19. The mobile inspection and handling system of claim 17, further comprising a camera system for visually inspecting multiple products and identifying a location of each unacceptable product.

20. The mobile inspection and handling system of claim 17, further comprising a location system for positioning the inspection and handling system in a specific location.

21. The method of claim 1, further comprising a step of securing the inspection and handling system relative to the conveyor with a male and female location system after it has been moved to a first location and subsequent locations.
